(19) United States
(12) Patent Application Publication
(10) Pub. No.: US 2009/0299230 A1
(43) Pub. Date: Dec. 3, 2009

(54) APPARATUSES, SYSTEMS, AND METHODS FOR MEASURING PORTIONS OF A BODY

(76) Inventor: Guy M. Houser, Bainbridge Island, WA (US)

Correspondence Address: PERKINS COIE LLP;PATENT-SEA, P.O. BOX 1247, SEATTLE, WA, 98111-1247, US

(21) Appl. No.: 12/496,345

(22) Filed: Jul. 1, 2009

Related U.S. Application Data
(62) Division of application No. 11/525,401, filed on Sep. 22, 2006, now abandoned.
(60) Provisional application No. 60/720,317, filed on Sep. 23, 2005.

(51) Int. Cl. A61B 5/103

(52) U.S. Cl. 600/587

(57) ABSTRACT

Apparatuses, systems, and methods for measuring portions of a body are disclosed herein. In one embodiment, an apparatus includes a disposable subassembly and a reusable subassembly releasably coupled to the disposable subassembly. The disposable subassembly includes a flexible member for positioning relative to the portion of the body. The reusable subassembly includes an encoder for detecting movement of the flexible member, a processor operably coupled to the encoder for receiving data corresponding to a dimension of the portion of the body, and an output device operably coupled to the processor for sending data to an external device.

### CROSS-REFERENCE TO RELATED APPLICATION

This application claims the benefit of U.S. Provisional Application No. 60/720,317, filed on Sep. 23, 2005, and entitled “APPARATUSES, SYSTEMS, AND METHODS FOR MEASURING PORTIONS OF A BODY,” which is incorporated by reference herein.

### TECHNICAL FIELD

The present disclosure is related to apparatuses, systems, and methods for measuring portions of a body.

### BACKGROUND

Burn victims typically wear compression garments over the injured tissue. The compression garments maintain a constant pressure on the injury that enables the tissue to heal in a more uniform manner and prevents hypertrophic and keloid scars, which can be painful as well as result in inflexible and unattractive skin. Compression garments can include fabric garments (e.g., sleeves and gloves), splints, or rigid plastic forms. Because the garments are required to maintain a generally uniform pressure over a relatively large area of tissue, multiple measurements of an individual must be taken to ensure that the garment properly fits the individual.

Conventional methods for measuring a bum victim include manually placing a tailor's tape on or around the portion of the victim to be measured, and then manually recording the measurement on a chart. This process is repeated numerous times to measure and record each measurement necessary for a particular compression garment. One drawback is that this process is slow because the individual taking the measurements must stop after each measurement and record the dimension on the chart. Another drawback is that this process is susceptible to errors. For example, the individual taking the measurements may misread the tape or transcribe an incorrect dimension. Another method for measuring burn victims includes scanning a portion of a victim with a laser scanner. Laser scanners, however, are relatively expensive.

### BRIEF DESCRIPTION OF THE DRAWINGS

FIG. 1 is a schematic illustration of a system for measuring a portion of a body in accordance with one embodiment of the invention.

FIG. 2A is a schematic isometric view of a mobile measuring device in accordance with one embodiment of the invention.

FIG. 2B is a schematic exploded view of the mobile measuring device of FIG. 2A.

FIG. 3 is a schematic isometric view of the reusable subassembly with the first portion of the first housing removed.

FIG. 4 is a schematic bottom view of the reusable subassembly.

FIG. 5 is a schematic exploded view of the removable subassembly.

FIG. 6 is a schematic top plan view of the removable subassembly with the first portion of the second housing removed.

FIG. 7 is a schematic isometric view of the mobile measuring device with the first portion of the first housing removed and the removable subassembly in the unlocked position.

FIG. 8 is a schematic isometric view of the mobile measuring device with the first portion of the first housing removed and the removable subassembly in the locked position.

FIG. 9 is a display diagram or a screenshot of one example of a tracking page for measurement data of the hands of the individual in accordance with one embodiment of the invention.

FIG. 10 illustrates an example of a sequence of measurements for measuring the dimensions of hands.

### DETAILED DESCRIPTION

### A. Overview

The following disclosure describes apparatuses, systems, and methods for measuring portions of a body. An embodiment of one such apparatus includes a disposable subassembly and a reusable subassembly releasably coupled to the disposable subassembly. The disposable subassembly includes a flexible member for positioning relative to the portion of the body. The reusable subassembly includes an encoder for detecting movement of the flexible member, a processor operably coupled to the encoder for receiving data corresponding to a dimension of the portion of the body, and an output device operably coupled to the processor for sending data to an external device.

In another embodiment, an apparatus includes a first housing, a flexible member positioned at least partially in the first housing, a second housing detachably coupled to the first housing, a processor positioned at least partially in the second housing, and an output device positioned at least partially in the second housing. The flexible member is configured for positioning relative to the portion of the body. The processor is configured for processing data based on at least one of the movement or position of the flexible member. The data corresponds to a dimension of the portion of the body. The output device is operably coupled to the processor for sending data corresponding to the dimension to an external device.

In another embodiment, an apparatus includes a first housing, a second housing removably attached to the first housing, means for measuring a dimension of a portion of a body, and means for sending the dimension to an external device. The means for measuring is positioned at least partially in at least one of the first or second housing. The means for sending is positioned at least partially in the first housing.

In yet another embodiment, a system for measuring a portion of a body includes a mobile measuring device having (a) a flexible member for positioning relative to the portion of the body, (b) a processor for processing data based on at least one of the movement or position of the flexible member and corresponding to a dimension of the portion of the body, and (c) an output device for sending data corresponding to the dimension of the portion of the body. The system further includes a computing system having a receiving device for receiving data from the output device and a storage device operably coupled to the receiving device for storing the received data.

In another embodiment, a method for measuring a portion of a body includes measuring a dimension of a portion of the body with a mobile measuring device, and transferring data corresponding to the dimension from the mobile measuring device to an external device.

In another embodiment, a method for measuring a portion of a body includes placing a flexible member at least proximate to the portion of the body, determining a dimension of the portion of the body based on at least one of the movement or position of the flexible member, and sending data corresponding to the dimension via a communications link to a computing device.

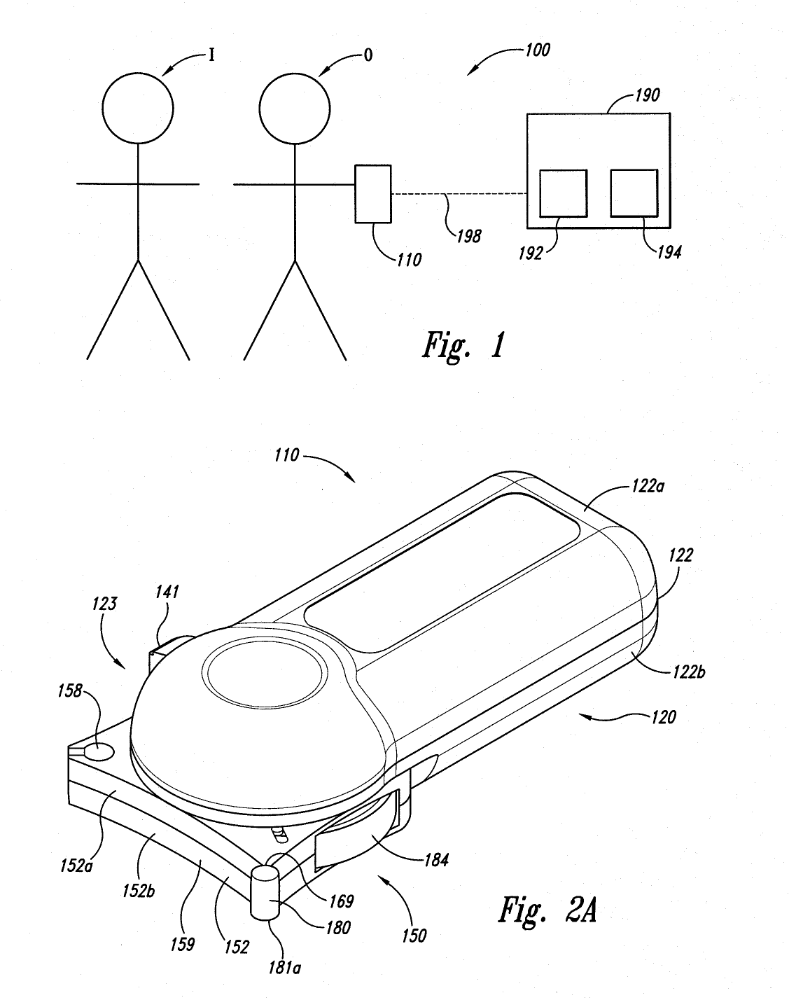

Certain details are set forth in the following description and in FIGS. 1-10 to provide a thorough understanding of various embodiments of the invention. Other details describing well-known structures and systems often associated with measurement devices are not set forth in the following disclosure to avoid unnecessarily obscuring the description of various embodiments of the invention. Many of the details, dimensions, angles, and other features shown in the figures are merely illustrative of particular embodiments of the invention. Accordingly, other embodiments can have other details, dimensions, and/or features without departing from the present invention. In addition, further embodiments of the invention may be practiced without several of the details described below, or various aspects of any of the embodiments described below can be combined in different embodiments. Where the context permits, singular or plural terms may also include the plural or singular term, respectively. Moreover, unless the word “or” is expressly limited to mean only a single item exclusive from other items in reference to a list of at least two items, then the use of “or” in such a list is to be interpreted as including (a) any single item in the list, (b) all of the items in the list, or (c) any combination of items in the list. The term “comprising” is used throughout to mean including at least the recited feature(s) such that any greater number of the same feature and/or types of other features or components are not precluded.

### B. Embodiments of Systems for Measuring Portions of a Body

FIG. 1 is a schematic illustration of a system 100 for measuring a portion of a body in accordance with one embodiment of the invention. The system 100 can be used by an operator O to measure one or more dimensions of an individual I. For example, the operator O can measure the circumference, diameter, length, angle, and/or another dimensions of the individual's hand, arm, foot, leg, torso, head, neck, and/or other portions of the body. The operator O may also measure the size of a wound or other feature on the individual I. Alternatively, the operator O can measure one or more dimensions of his or her own body. In either case, the measurements can be used to manufacture clothing, garments, footwear, helmets, accessories, prosthetic limbs, and/or other articles that require one or more measurements of the body. For example, the measurements can be used to manufacture custom compression garments with a precise size for patients recovering from burns. The measurements can also be used to determine if a prescribed treatment is effective. For example, the size of a wound or other abnormality on the body can be measured at different times during treatment to determine the effect of the treatment on the wound or other abnormality.

The illustrated system 100 includes a mobile measuring device 110 and a computing system 190 spaced apart and separate from the measuring device 110. The mobile measuring device 110 can be a handheld unit that measures one or more dimensions of a body and transfers data corresponding to the dimensions to the computing system 190 via a communications link 198. The communications link 198 can be a wireless connection (e.g., infrared, radio frequency, WiFi, Bluetooth) or a wired connection (e.g., USB, serial or parallel ports). The computing system 190 includes a receiving device 192 for receiving data via the communications link 198 and a storage device 194 for storing the data. Depending on the type of communications link 198, the receiving device 192 can include a wireless receiver, a USB port, or another suitable device for receiving data from the mobile device 110.

### C. Embodiments of Apparatuses for Measuring Portions of a Body

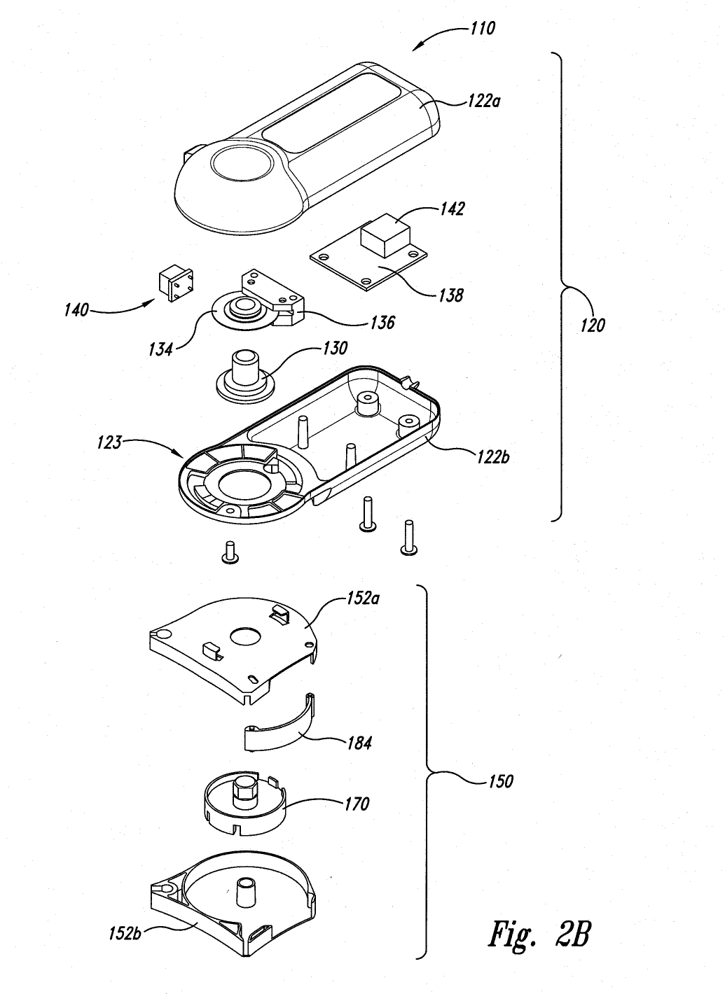

FIG. 2A is a schematic isometric view and FIG. 2B is a schematic exploded view of the mobile measuring device 110 in accordance with one embodiment of the invention. Referring to both FIGS. 2A and 2B, the illustrated measuring device 110 includes a reusable subassembly 120 and a removable subassembly 150 detachably coupled to the reusable subassembly 120. The reusable subassembly 120 includes a first housing 122 with a first portion 122a and a second portion 122b attached to the first portion 122a. Although the illustrated first housing 122 has a shape generally similar to a computer mouse, in other embodiments the first housing 122 can have other ergonomic shapes. The removable subassembly 150 includes a second housing 152 with a first portion 152a and a second portion 152b attached to the first portion 152a. The first portion 152a of the second housing 152 is detachably coupled to the second portion 122b of the first housing 122 at a first end section 123 of the first housing 120. Although the second housing 152 has a generally different contour than the first housing 122, in other embodiments the first and second housings 122 and 152 can have one or more generally continuous surfaces.

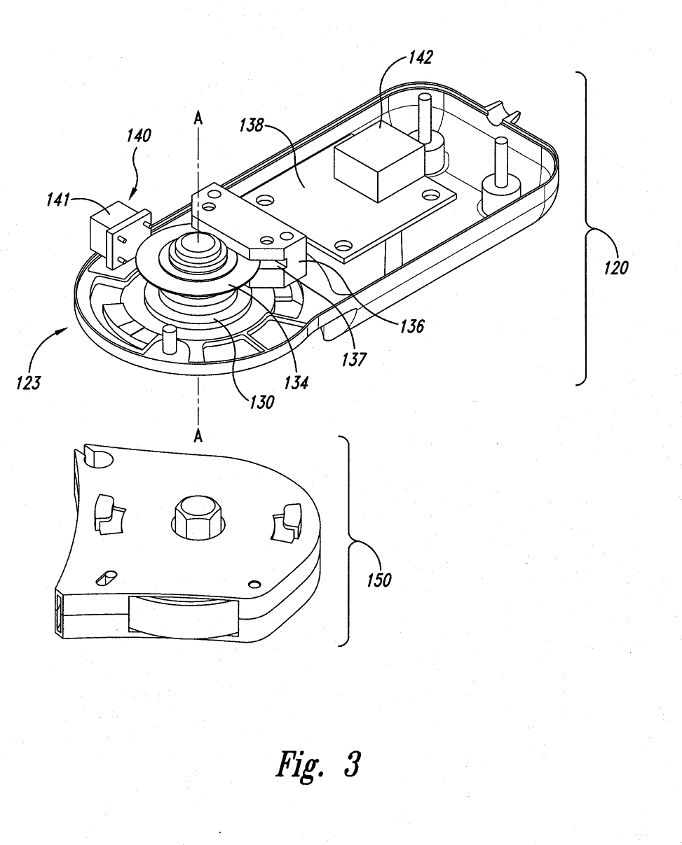

FIG. 3 is a schematic isometric view of the reusable subassembly 120 with the first portion 122a of the first housing 122 removed. The illustrated reusable subassembly 120 includes a hub 130 positioned at the first end section 123 of the first housing 122, a disk 134 carried by the hub 130, and an encoder 136 positioned adjacent to the disk 134. Specifically, the disk 134 is attached to the hub 130 so that the disk 134 and the hub 130 rotate together about an axis A-A. The encoder 136 includes a slot 137 positioned to receive a portion of the disk 134 and measure the movement of the disk 134 and the hub 130. For example, the encoder 136 can be an optical encoder, and the disk 134 can include optical marks for the optical encoder to detect. In other embodiments, however, the reusable subassembly 120 may not include the disk 134 and/or the encoder 136, but rather may include a different device to measure movement of the hub 130.

The illustrated reusable subassembly 120 further includes a battery (not shown) or other power source, a processor 138, a switch 140 for instructing the processor 138 to capture data from the encoder 136, and an output device 142 for sending data from the processor 138 to the computing system 190 (FIG. 1) or another external device. Although the illustrated processor 138 is a USB processor 190 and the illustrated output device 142 is a USB port, in other embodiments, the processor 138 can include a serial processor, a parallel processor, or another type of processor, and the output device 142 can include a serial port, a parallel port, an infrared transmitter, a radio-frequency transmitter, a Bluetooth transmitter, a WiFi transmitter, or another suitable device for transmitting data to the external device. The illustrated switch 140 includes a depressible button 141 for the operator O (FIG. 1) to press for instructing the processor 138 to capture data from the encoder 136. In additional embodiments, the switch 140 may not include the button 141, but rather a different device for the operator O to manipulate. In embodiments in which the reusable subassembly 120 includes a wireless transmitter, the subassembly 120 may also include a receiver to receive data from the computing system 190.

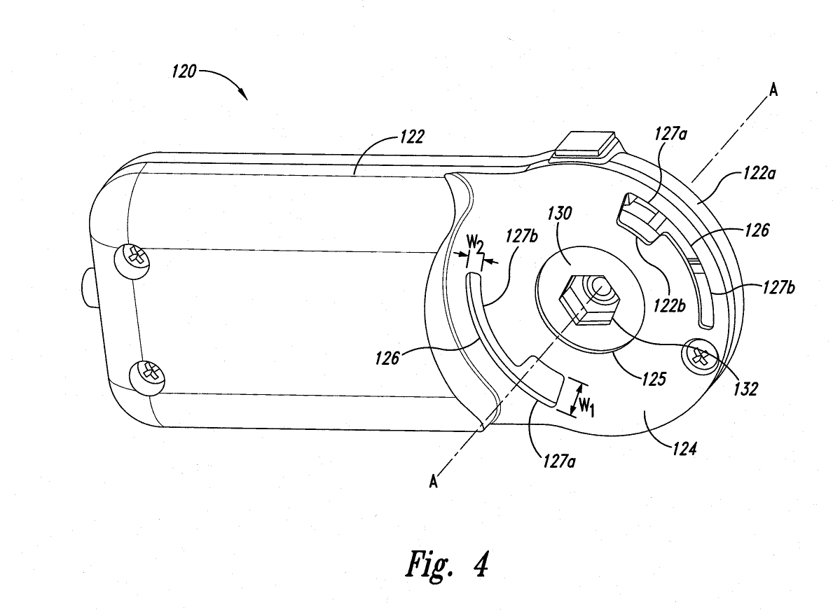

FIG. 4 is a schematic bottom view of the reusable subassembly 120. The illustrated second portion 122b of the first housing 122 includes a recessed surface 124 at the first end section 123, an aperture 125 in the recessed surface 124, and two arcuate slots 126 in the recessed surface 124. The aperture 125 is aligned with the axis A-A and exposes an aperture 132 in the hub 130. The two arcuate slots 126 are positioned radially outward of the aperture 125 and are spaced apart circumferentially. The illustrated slots 126 include a first portion 127a with a first radial width W₁ and a second portion 127b with a second radial width W₂ less than the first radial width W₁. The slots 126 are configured to receive corresponding connectors of the removable subassembly 150 so that the reusable subassembly 120 can be detachably coupled to the removable subassembly 150.

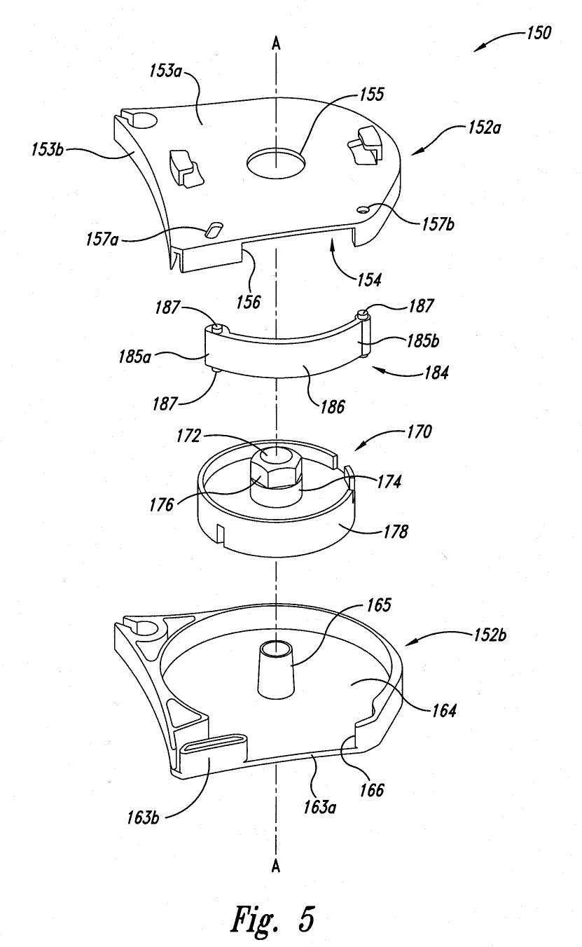

FIG. 5 is a schematic exploded view of the removable subassembly 150. The first portion 152a of the second housing 152 includes a base 153a, a sidewall 153b projecting from the base 153a, a recess 154 defined by the base 153a and the sidewall 153b, and an aperture 155 in the base 153a generally aligned with the axis A-A. The second portion 152b of the second housing 152 includes a base 163a, a sidewall 163b projecting from the base 163a, a recess 164 defined by the base 163a and the sidewall 163b, and a post 165 projecting from the base 163a and generally aligned with the axis A-A. The recesses 154 and 164 form a chamber to receive other components of the removable subassembly 150.

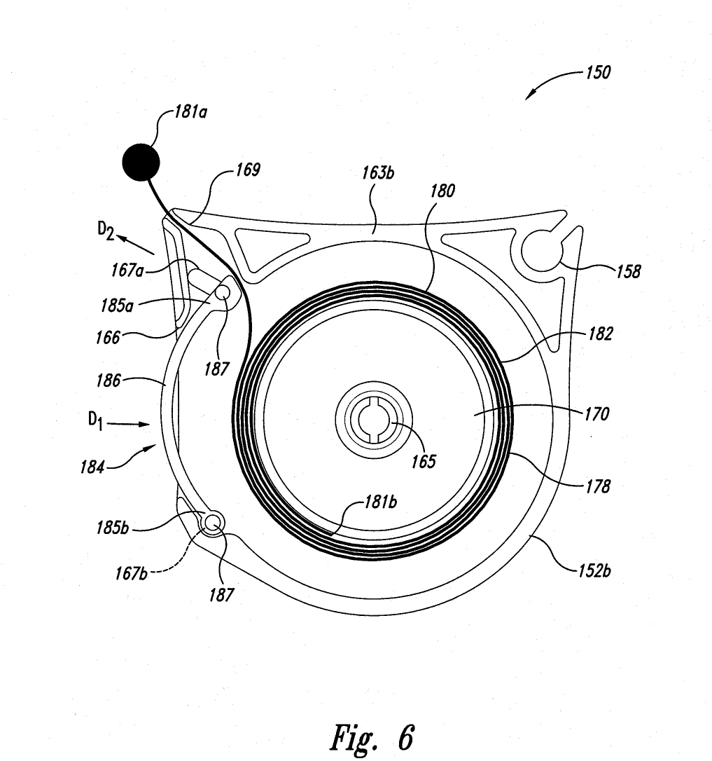

The illustrated removable subassembly 150 further includes a spool 170 positioned in the recesses 154 and 164, a flexible member 180 (shown in FIG. 6) at least partially wound around the spool 170, and a release member 184 partially positioned within the second housing 152. The spool 170 has an aperture 172 generally aligned with the axis A-A, a projection 174 generally aligned with the axis A-A, and an external surface 178 around which the flexible member 180 is wound. The aperture 172 is sized to receive. the post 165 of the second portion 152b such that the spool 170 can rotate freely relative to the second portion 152b as the flexible member 180 pays out from or is retracted in the second housing 152. The projection 174 includes a distal portion 176 sized to extend through the aperture 155 in the first portion 152a and project from the second housing 152 as seen in FIG. 3. The distal portion 176 is also shaped and sized to correspond to the aperture 132 (FIG. 4) in the hub 130 so that when the removable subassembly 150 is attached to the reusable assembly 120, the distal portion 176 is received in the aperture 132 and the hub 130 and spool 170 rotate together about the axis A-A. As a result, when the flexible member 180 pays out from or is retracted into the second housing 152, the flexible member 180 rotates the spool 170, which drives the hub 130, which in turn rotates the disk 134 (FIG. 3). As described above, the encoder 136 measures the rotation of the disk 134 and, consequently, the movement of the flexible member 180.

In additional embodiments, the encoder 136 may not measure the movement of the flexible member 180 via the spool 170, hub 130, and disk 134. For example, in one embodiment, a rotary wheel can be attached to the second housing 152 proximate to the channel 169 and positioned so that the flexible member 180 rotates the rotary wheel as the member 180 pays out from and is retracted back into the second housing 152. A rotary encoder can be operably coupled to the rotary wheel to measure the rotation of the wheel. In yet another embodiment, optical indexing marks can be placed on the flexible member 180 and an encoder can be positioned to detect the marks on the flexible member 180 as the member 180 pays out from and is retracted back into the second housing 152.

FIG. 6 is a schematic top plan view of the removable subassembly 150 with the first portion 152a of the second housing 152 removed. The flexible member 180 includes a first, free end 181a positioned outside the second housing 152, a second end 181b opposite the first end 181a and attached to the spool 170, and an intermediate section 182 extending between the first and second ends 181a-b. The flexible member 180 can be a tape, cloth or vinyl strip, cable, string, or other flexible member that at least generally conforms to the contour of a body. The illustrated second housing 152 also includes a channel 169 for receiving the flexible member 180 and a slot 158 corresponding to the shape and size of the first end 181a of the flexible member 180. In other embodiments, the second housing 152 may not include the slot 158 and the first end 181a of the flexible member 180 can include a hook or other device for releasably connecting the first end 181a to the intermediate section 182 when the flexible member 180 is wrapped around a portion of a body.

Referring to both FIGS. 5 and 6, the first portion 152a (FIG. 5) of the second housing 152 further includes a slot 157a in the base 153a, an aperture 157b in the base 153a, and an opening 156 in the sidewall 153b. The second portion 152b (FIG. 6) of the second housing 152 further includes a slot 167a in the base 163a, an aperture 167b in the base 163a, and an opening 166 in the sidewall 163b. The slots 157a and 167a, apertures 157b and 167b, and openings 156 and 166 are sized to receive portions of the release member 184. Specifically, the illustrated release member 184 includes a first end portion 185a, a second end portion 185b opposite the first end portion 185a, and an intermediate portion 186 extending between the first and second end portions 185a-b. The first and second end sections 185a-b include projections 187 sized to be inserted into corresponding slots 157a and 167a and apertures 157b and 167b in the first and second portions 152a-b. The intermediate portion 186 is sized and positioned to extend through the openings 156 and 166 and project from the second housing 150.

Referring only to FIG. 6, the release member 184 selectively allows the flexible member 180 to pay out from and retract back into the second housing 152. For example, the illustrated release member 184 is movable between a lock position (shown in FIG. 6) and a release position (not shown). In the lock position, the first end portion 185a presses the flexible member 180 against the sidewalls 153b and 163b of the first and second portions 152a-b to inhibit movement of the flexible member 180. To move the release member 184 from the lock position to the release position, the operator O (FIG. 1) presses the intermediate section 186 in a direction D₁, which moves the first end portion 185a in a direction D₂ through the slots 157a (FIG. 5) and 167a (FIG. 6) such that the first end portion 185a does not press the flexible member 180 against the sidewalls 153b and 163b. Thus, in the release position, the flexible member 180 can pay out from or retract back into the second housing 150. The removable subassembly 150 can further include a rotary spring (not shown) or other urging member to urge the spool 170 to retract the flexible member 180. In other embodiments, the removable subassembly 150 can include another device with a different configuration than the release member 184 for controlling the movement of the flexible member 180.

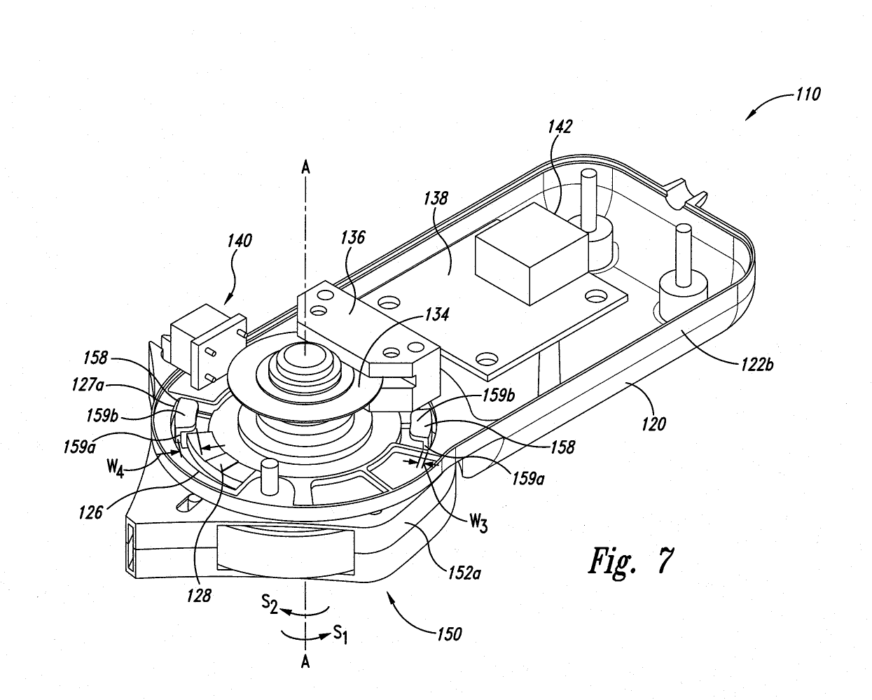

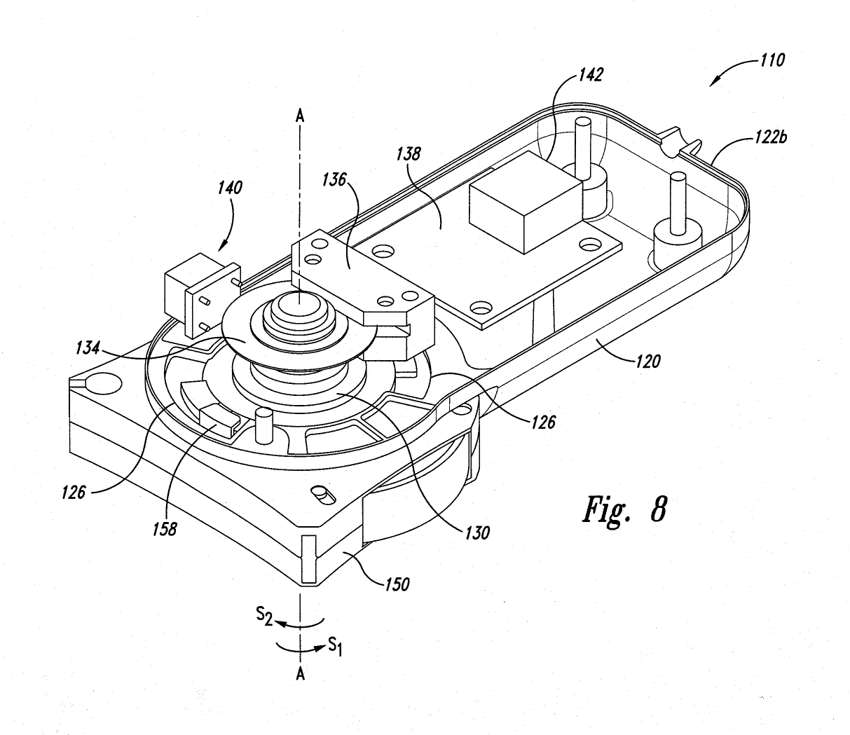

FIG. 7 is a schematic isometric view of the mobile measuring device 110 with the first portion 122a of the first housing 122 removed. The removable subassembly 150 further includes two connectors 158 on the first portion 152a of the second housing 152 for releasably connecting the removable subassembly 150 to the reusable subassembly 120. The connectors 158 are positioned and sized to be received in the slots 126 of the second portion 122b of the first housing 122. Specifically, the illustrated connectors 150 include a first portion 159a projecting in a direction generally parallel to the axis A-A, and a second portion 159b projecting radially inward from the first portion 159a. The individual second portions 159b have a radial width W₄ less than the radial width W₁ (FIG. 4) of the first portion, 127a of the slots 126 so that the connectors 158 can be inserted into the first housing 122. The individual first portions 159a have a radial width W₃ less than the radial width W₂ (FIG. 4) of the second portion 127b of the slots 126 so that the removable subassembly 150 can be pivoted in a direction S₁ about the axis A-A and move from a detachment position (illustrated in FIG. 7) to a lock position (illustrated in FIG. 8), in which the removable subassembly 150 is attached to the reusable subassembly 120. The second portion 122b of the first housing 122 may also include detents 128 positioned to engage the second portion 159b of the connectors 158 to inhibit inadvertent rotation of the removable subassembly 150 in a direction S₂ about the axis A-A.

In additional embodiments, the mobile measuring device 110 may include a display for providing information to the operator O and/or the individual I. For example, the display may provide instructions to the operator O that indicates which measurement to take, the dimension of a particular measurement, and/or other information. The display may also provide an indication of whether the measuring device 110 is connected to the computing system 190. In other embodiments, the mobile measuring device 110 may include other features in lieu of or in addition to the features described above with reference to FIGS. 1-8.

### D. Embodiments of Methods for Measuring Portions of a Body

Referring to FIGS. 1 and 2A, in operation, the operator O can use the mobile measuring device 110 to measure one or more dimensions of the individual I. For example, the operator O can measure the circumference of the individual's wrist by pressing the release member 184, pulling a section of the flexible member 180 out of the second housing 152 while pressing the release member 184, wrapping the flexible member 180 around the individual's wrist, and positioning the measuring device 110 so that the section of the flexible member 180 that is not wrapped around the individual's wrist is retracted into the second housing 152. When the measuring device 110 is properly positioned, the operator O presses the button 141 and the switch 140 instructs the processor 138 to capture the data on the encoder 136 corresponding to the dimension of the wrist. The data is then transferred to the computing system 190 via the communications link 198. Alternatively, the data can be sent to the computing system 190 after multiple measurements have been taken. In several embodiments, the data represents the rotation of the spool 170, and the computing system 190 calculates the distance the flexible member 180 paid out from the second housing 152 based on the data. The calculation can incorporate the change in the diameter of the flexible member 180 wound around the spool 170 as the flexible member 180 pays out from and is retracted into the second housing 152. In other embodiments, the processor 138 in the measuring device 110 can calculate the distance the flexible member 180 pays out from and is retracted into the second housing 152, and then send the data to the computing system 190.

In another embodiment of a method for measuring the circumference of the individual's wrist, the operator O presses the release member 184, pulls a section of the flexible member 180 out of the second housing 152 while pressing the release member 184, wraps the flexible member 180 around the wrist, places the first end 181a of the flexible member 180 in the slot 158 of the second housing 152, and positions the device 110 so that a surface 159 of the second housing 152 contacts the wrist. After the measuring device 110 has been properly positioned, the operator O presses the button 141 and the switch 140 instructs the processor 138 to capture the data on the encoder 136. In this particular embodiment, the operator O presses the button twice when the first end 181a of the flexible member 180 is received in the slot 158 to instruct the processor 138 to incorporate the offset distance between the channel 169 and the slot 158 into the calculation of the dimension. In other embodiments, however, other methods or means can be used to instruct the processor 138 to incorporate the offset distance between the channel 169 and the slot 158 into the dimension calculation.

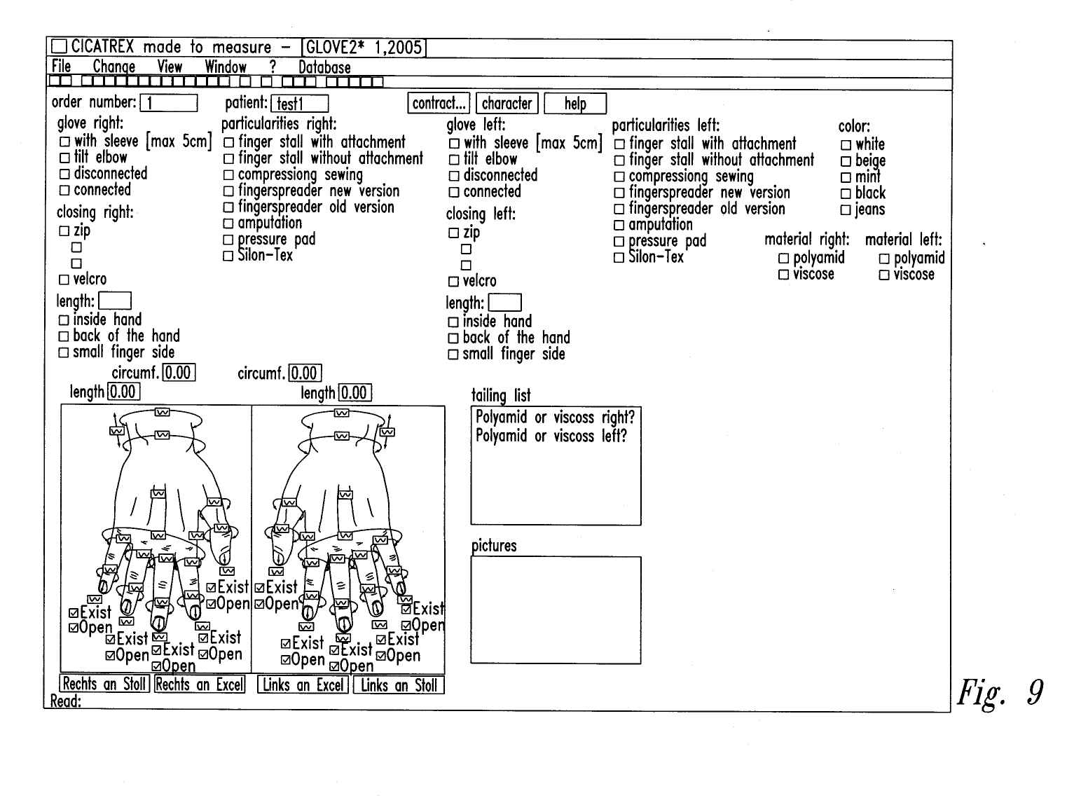

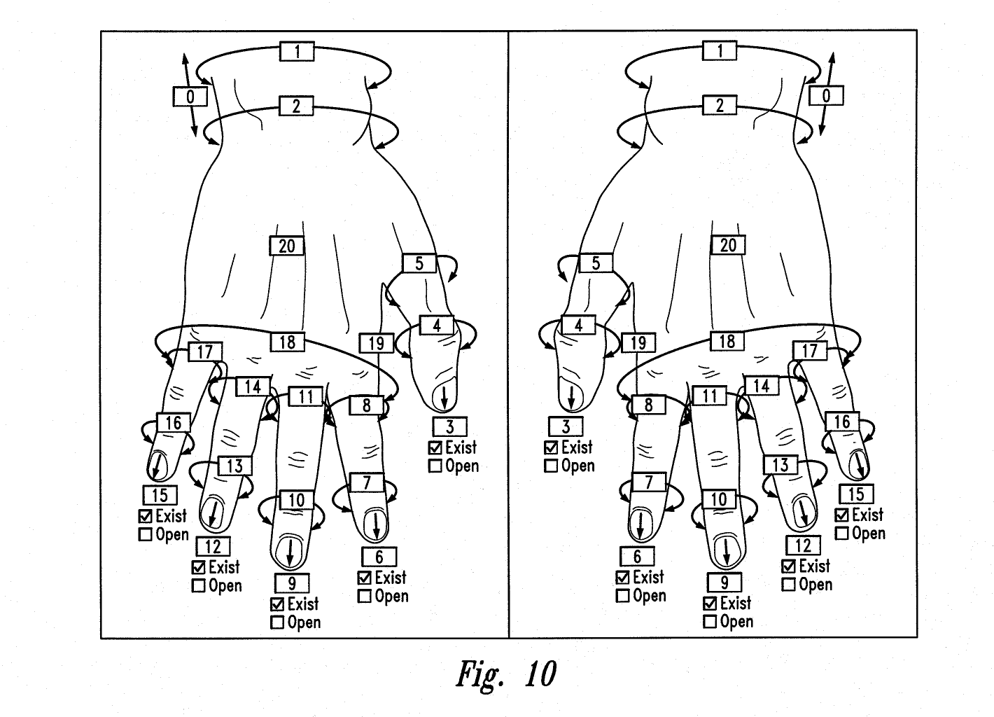

Referring only to FIG. 1, in several embodiments, the computing system 190 includes and/or runs an application to track the data from the measuring device 110. For example, FIG. 9 is a display diagram or a screenshot of one example of a tracking page for measurement data of the hands of the individual I. The illustrated screenshot shows the operator O all the measurements that are required for a specific application. If multiple measurements are needed, the computing system 190 may require the operator O to take the measurements in a specific sequence so that the data is associated with the proper measurements. The sequence may be selected to maximize efficiency and/or minimize movement of the individual I. FIG. 10 illustrates an example of a sequence of measurements for measuring the dimensions of hands. In several embodiments, the computing system 190 may provide audible prompts to the operator O regarding the sequence of the measurements. For example, after the operator O measures the circumference of the index finger at the joint between the middle and distal phalanges (labeled as measurement 7 in FIG. 10) and transfers the data to the computing system 190, the system 190 may provide an audible instruction to the operator O that the next required measurement is the circumference of the index finger at the base of the finger (labeled as measurement 8 in FIG. 10). In other embodiments, the computing system 190 may not provide audible prompts and/or require that measurements be taken in a specific sequence. For example, the mobile device and/or computing system may include an input device through which the operator can input the particular measurement taken.

In several embodiments, the computing system 190 can also perform a quality control check for each measurement. For example, the computing system 190 may include a predetermined acceptable range for each measurement. As a result, if a specific measurement falls outside the acceptable range, the computing system 190 may request a confirmation from the operator O that the proper measurement was taken and the dimension is accurate. Alternatively, the computing system 190 may provide some other indication that the measurement falls outside the predetermined acceptable range.

One feature of the system 100 illustrated in FIGS. 1-10 is that the mobile device 110 automatically transfers the data corresponding to the measurements to the computing system 190. An advantage of this feature is that the measuring process is quicker and more efficient because the operator O does not need to manually record the measurements on a chart or manually enter the measurements into a computer. Rather, the operator O can take one measurement, transfer the data to the computing system 190, and take the next measurement without moving away from the individual I. This enables the operator O to focus his or her attention on the individual, which may enhance the experience of the individual I. Another advantage of this feature is that the data is likely to be more accurate because it is not subject to manual recording and input errors.

Another feature of the mobile measuring device 110 illustrated in FIGS. 1-8 is that a first removable subassembly 150 can be detached from the device 110 after use with one individual and a second removable subassembly 150 can be attached to the device 110 for use with another individual. An advantage of this feature is that the components of the device 110 that may come into contact with the individual (e.g., the flexible member 180 and the second housing 152) can be discarded after use with a single individual and the more expensive components of the device 110 (e.g., the encoder 136 and the processor 138) can be reused. This is expected to significantly reduce the costs associated with measuring a portion of an individual.

One feature of an embodiment of the system 100 in which the mobile device 110 and the computing system 190 communicate via a wireless link is that the mobile device 110 and the computing system 190 can be placed in different rooms and/or separated by a wall or other barrier. An advantage of this feature is that the mobile device 110 can be sterilized and used in operation rooms, emergency rooms, and/or other locations in which objects must be sterile. The computing system 190 can be placed in an adjacent room such that the computing system 190 need not be sterilized. This is expected to reduce the costs of operating the system 100. In other embodiments, however, the computing system 190 can be sterilized and/or placed in the same room as the mobile device 110.

From the foregoing, it will be appreciated that specific embodiments of the invention have been described herein for purposes of illustration, but that various modifications may be made without deviating from the invention. For example, although the illustrated measuring device 110 includes a removable subassembly 150 that can be discarded after a single use, in other embodiments, the subassembly 150 may be reused and may not be discarded after one or more uses. Moreover, although the above text describes methods for measuring human bodies, the illustrated system 100 can be used to measure animals, inanimate objects, and/or other structures. For example, the system may measure the dimensions of a horse to provide a properly sized saddle. Furthermore, aspects of the invention described in the context of particular embodiments may be combined or eliminated in other embodiments.

1-22. (canceled)

23. A method for measuring a portion of a body, the method comprising:
measuring a dimension of a portion of the body with a mobile measuring device; and
transferring data corresponding to the measured dimension from the mobile measuring device to an external device.

24. The method of claim 23 wherein transferring data corresponding to the measured dimension comprises transferring the data from the mobile measuring device to the external device via a wireless link.

25. The method of claim 23, further comprising disposing of a first portion of the mobile measuring device and reusing a second portion of the mobile measuring device.

26. The method of claim 23, further comprising detaching a first portion of the mobile measuring device from a second portion of the mobile measuring device after transferring data to the external device.

27. The method of claim 23 wherein measuring the dimension of the portion of the body comprises measuring a first dimension of the portion of the body, wherein transferring data corresponding to the measured dimension comprises transferring data corresponding to the first measured dimension, and wherein the method further comprises:
measuring a second dimension of the portion of the body with the mobile measuring device after transferring data corresponding to the first measured dimension; and
transferring data corresponding to the second measured dimension from the mobile measuring device to the external device.

28. The method of claim 23 wherein transferring data corresponding to the measured dimension comprises transferring the data from the mobile measuring device to the external device via a wired connection.

29. A method for measuring a portion of a body, the method comprising:
placing a flexible member at least proximate to the portion of the body;
automatically determining a dimension of the portion of the body based on at least one of the movement or position of the flexible member; and
sending data corresponding to the dimension via a communications link to a computing device.

30. The method of claim 29, further comprising associating the received data with a particular measurement of the body at the computing device.

31. A method for measuring one or more body parts of one or more persons, comprising:
withdrawing a first flexible member from a first disposable subassembly relative to a body part of a first patient;
determining a distance that first flexible member is withdrawn from the first disposable subassembly using an encoder within a reusable subassembly, wherein the reusable subassembly is releasably attached to the first disposable subassembly;
detaching the first disposable subassembly from the reusable subassembly after measuring the body part of the first patient;
attaching a second disposable subassembly to the reusable subassembly;
withdrawing a second flexible member from the second disposable subassembly relative to a body part of a second patient; and
determining a distance that the second flexible member is withdrawn from the second disposable subassembly using the encoder within the reusable subassembly.

32. The method of claim 31 wherein:
the first flexible member comprises a first tape wrapped around a first spool in the first disposable subassembly, the second flexible member comprises a second tape wrapped around a second spool in the second disposable subassembly, and the first and second tapes have optical marks;
the encoder comprises an optical sensor capable of sensing the optical marks; and
determining the distance that the first and second flexible members are withdrawn from the first and second disposable subassemblies, respectively, comprises counting the optical marks on the first and second tapes using the encoder.

33. The method of claim 31 wherein:
the first flexible member comprises a first tape wrapped around a first spool in the first disposable subassembly and the second flexible member comprises a second tape wrapped around a second spool in the second disposable subassembly;
the encoder comprises a disk and an optical sensor capable of sensing rotation of the disk, wherein the disk is releasably coupled to the first and second tapes such that the disk rotates as the first and second tapes are withdrawn from the first and second disposable subassemblies; and
determining the distance that the first and second flexible members are withdrawn from the first and second disposable subassemblies comprises monitoring rotation of the disk using the optical sensor.

34. The method of claim 31, further comprising:
detaching the second disposable subassembly from the reusable subassembly after measuring the body part of the second patient;
attaching a new disposable subassembly to the reusable subassembly;
withdrawing a flexible member from the new disposable subassembly relative to a body part of another patient; and
determining a distance that the flexible member is withdrawn from the new disposable subassembly using the encoder within the reusable subassembly.

35. The method of claim 31, further comprising automatically determining a dimension of the body parts of the first and second patients based on the determined distances that the first and second flexible members are withdrawn from the first and second disposable subassemblies, respectively.

36. The method of claim 35, further comprising sending data corresponding to the determined dimensions of the body parts of the first and second patients to a computing device.
